SEQUENCE LISTING (3 sequences)

SEQ ID NO: 1 - protein, 9 aa, Human papillomavirus type 16
YIIFVYIPL

SEQ ID NO: 2 - protein, 9 aa, artificial
  tyrosinase peptide 369-377 (positive control)
YMDGTMSQV

SEQ ID NO: 3 - protein, 9 aa, artificial
  melanoma antigen-1 peptide 161-169(negative control)
EADPTGHSY

(19) United States
(12) Patent Application Publication
(10) Pub. No.: US 2007/0218080 A1
(43) Pub. Date: Sep. 20, 2007

(54) Peptide antigen of human papillomavirus type 16 and application thereof

(75) Inventors: Yuh-Cheng Yang, Taipei City (TW); Yeou-Ping Tsao, Taipei City (TW); Show-Li Chen, Taipei City (TW)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: Mackay Memorial Hospital, Taipei City (TW)

(21) Appl. No.: 11/375,226

(22) Filed: Mar. 14, 2006

(51) Int. Cl. C12Q 1/68; A61K 39/12; C07K 14/00; C07K 16/00; A61K 38/00; C07K 17/00; C07K 2/00; C07K 4/00; C07K 5/00; C07K 7/00

(52) U.S. Cl. 424/204.1; 530/300; 435/6

(57) ABSTRACT

Disclosed is a HPV-16 peptide antigen, and the antigen comprises a peptide sequence of SEQ ID No: 1. The peptide antigen of the invention is able to bind with MHC class I antigen on the cell and be presented on the cell surface. It can be recognized by cytotoxic T lymphocytes (CTLs), therefore induces CTL responses for cytolysis to control the replication and expression of the HPV-16.

### BACKGROUND OF THE INVENTION

1. Field of the Invention

The present invention relates to a viral antigen of human papillomavirus (HPV), especially relates to a peptide antigen of HPV type 16 (HPV-16) E5 protein.

2. The Prior Arts

Cervical cancer accounts for 12% of all cancers in women, which is the second most frequent malignancy in women of the world, represents a great health threat to women. According to the recent research, more than 90% cases of cervical cancer are caused by human papillomavirus (HPV) infections. Among more than 90 types of the known HPV, there are more than 30 high-risk HPV types, which are associated with the cancers of female reproductive organs. Among these viruses, HPV genotype 16 (abbreviated HPV-16 hereafter) is most closely related to cervical cancer since HPV-16 were found in more than 60% of the cervical cancer patients.

The genome of HPV-16 consists of at least 8 open reading frames such as E1˜E7, L1 and L2. E5, E6 and E7 are oncogenes encoding oncoproteins to cause malignant transformation of cells. HPV-15 E5 protein stimulates cell growth continuously and oncogenesis by forming a complex with the epidermal growth-factor receptor, platelet-derived growth factor receptor or colony-stimulating factor-1 receptor. On the other hand, E5 protein induces T-cell mediated immune responses, stimulates the proliferation of cytotoxic T lymphocytes (CTLs), and kills the infected cells through cytotoxic lysis to further control the proliferation and expansion resulted by HPV E5 protein.

The role of E5 protein in T-cell mediated immunity is discussed below. Generally E5 protein will be processed into several peptide fragments in lengths of 8-11 amino acids after virus enters the cell. Part of the peptide fragments can bind major histocompatability complex class I (abbreviated MHC class I hereafter) antigen in endoplasmic reticulum, and display on the infected cell surface. The receptors in the surfaces of CTLs (also known as CD8 lymphocytes) recognize the antigens displaying on the infected cell surface after E5 protein are bound. Therefore if CD8 lymphocytes are able to recognize the viral peptides when viral E5 protein are bound to the surface of MHC class I antigens, CD8 lymphocytes will perform cytotoxicity and kill the infected cells. Therefore, lysis of infected cells after recognition depends on E5 protein-specific CD8 lymphocytes.

Though E5 protein induces the immune responses of the infected cells, on the other hand, it could also loss control on cell proliferation (such as oncogenesis). Therefore, the MHC class I restricted epitope of E5 protein in cell surface needs to be identified if E5 protein is used in controlling the growth of cancer cells. And the peptide fragments containing this epitope should be used to elicit immune responses to prevent the oncogenic potential induced by full E5 protein.

Since E5 protein is known to exist in patients with early-stage cervical cancer, vaccination with E5 protein will build great effects in early prevention or controlling against the HPV-16 associated cervical cancer. Therefore, further studies on epitope of E5 protein amino acid sequence for vaccination will provide women the best protection on HPV prevention.

### SUMMARY OF THE INVENTION

A primary object of the present invention is to provide an oligopeptide on E5 protein of HPV-16, which can induce CTL responses for cytolysis and through the amino sequence of the peptide to provide vaccination on prevention against the HPV-16 associated cervical cancer or the medical composition toward early stage cancer.

The HPV-16 peptide antigen according to the present invention comprises a amino acid sequence of SEQ ID NO: 1. Amino acid sequence SEQ ID NO: 1 of the invention is the same as the 63th to 71th amino acid sequence of E5 protein. The peptide sequence identified in the invention can bind the MHC class I tissue antigens (such as Human Leucocyte Antigen HLA-A2.1) and be presented on the cell surface after binding, therefore stimulates the cytolytic activity of CTLs.

The HPV-16 peptide antigen identified in the present invention can be produced by the known peptide synthesis technology or instruments according to the peptide of the invention, or obtained from translation of the E5 oncogene, or from digestion of the natural E5 protein.

The peptide antigen of the invention can be applied in vaccine preparation. The proliferation of CD8 lymphocytes against the peptide antigen is induced through injection of this peptide vaccine of the invention. When the HPV-16 viruses attack human body, CD8 lymphocytes proliferated is able to recognize and destroy E5 protein containing HPV-16 infected cells to protect against development of cancer.

On the other hand, because E5 protein antigen is expressed in early stage of HPV-16 infection in hosts, peptide of SEQ ID NO: 1 in the invention can be used as antigen to induce CD8 lymphocytes, to lyse the cells in virus's associated pre-cancerous condition, reduce the occurrence of malignant neoplasm and therefore effectively prevent or control HPV-16 associated cervical cancer.

The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

### BRIEF DESCRIPTION OF THE DRAWINGS

FIG. 1 shows FITC fluorescence intensities of SEQ ID NO: 1 group, positive control group, and negative control group using flow cytometry assay.

FIG. 2 shows the proliferation of CD8 cells induced by SEQ ID NO: 1 peptide in comparison to the control group using flow cytometry assay; (A): experimental group; (B) control group.

FIG. 3 shows the CTL activities of SEQ ID NO: 1 peptide after immunization at various effector-to-target cell ratio in vitro; black circle represents an experimental group incubating target cells with effector cells at various ratios; and empty diamond represents a control group incubating target cells in the absence of effector cells.

### DETAILED DESCRIPTION OF THE PREFERRED EMBODIMENT

### EXAMPLE 1

### T2 Cell Binding Assays

Human Leukocyte Antigen-A2.1 (abbreviated HLA-A2.1 hereafter) belonging to human MHC class I tissue antigen were used to screen peptides of E5 protein which could bind human MHC I tissue antigen. HLA-A2.1 antigen is a common HLA allele in people, which presents in more than 50% of the population. Firstly, a HLA peptide binding prediction program offered by the National Institute of Health (NIH) (http://www.bimas.dcrt.nih.gov/cgi-bin/molbio/hla-bind/) in length of 9-amino acids was used to predict the potential HLA-A2.1 binding peptide sequences for confirmation experiments followed.

Through the abovementioned program, a peptide sequence was identified in E5 peptide 63-71 as shown in SEQ ID NO: 1. This peptide is synthesized by conventional solid-phase peptide synthesis using an Abimed AMS 422 peptide synthesizer. The products were purified with a reverse-phase high-performance liquid chromatography (HPLC) and lyophilized. Finally the synthesized peptidse were dissolved in phosphate buffer saline (PBS) with less than 1% dimethyl sulfoxide (DMSO) in a concentration of 5 mg/ml.

Another group of HLA-A2.1 binding peptides of SEQ ID NO: 2 was prepared as the positive control. Peptide SEQ ID NO: 2 is obtained from tyrosinase peptide 369-377. Peptide of SEQ ID NO: 3 binding to HLA-A1 but not HLA-A2.1 was employed as a negative control. Peptide SEQ ID NO: 3 is obtained from melanoma antigen-1 peptide 161-169. Both the peptides in positive and negative control groups were prepared in the same way as described, also dissolved in the same solution at the same concentration of the experimental group.

The binding test of the peptide in the invention was subjected to a T2 cell binding assay to measure the binding affinity for HLA-A2.1 antigens on T2 cell surface. T2 cells lack genes coding for transporter proteins for antigenic peptides (TAP1/TAP2) and therefore is not able to translocate peptide antigens processed by cells into the endoplasmic reticulum (ER). The antigens will not be displayed, and the assays will be interfered. Therefore, the synthetic peptide antigens were used directly, not through cell processing, for the binding test with HLA-A2.1 antigens on T2 cell surface.

T2 cells that had been incubated overnight at 26° C. in RPMI 1640 containing 5% fetal bovine serum (FBS) were mixed with 10 μg/ml of pre-prepared synthetic peptide for 20 hours at 26° C. in RPMI containing 5% FCS and 5 μg/ml human β 2-microglobulin (β 2 m). The cells were then washed twice, incubated at 37° C. for 2 hours. If the HLA-A2.1 antigens did not bind synthetic peptides, these HLA-A2.1 antigens will be buried inside the cell membrane and not be displayed. Then the cells were incubated with fluorescein isothiocyanate (FITC) labeled anti-human HLA-A2.1 antibodies (BD Biosciences Pharmingen) to stain HLA-A2.1. The stains were fixed with 1% paraformaldehyde, and analyzed by flow cytometry. Because the binding of synthetic peptides and HLA-A2.1 antigens would be presented on the surface of lymphocytes, which could be recognized and bound by anti-human HLA-A2.1 antibodies. Therefore the fluorescein indicated the binding of synthetic peptides.

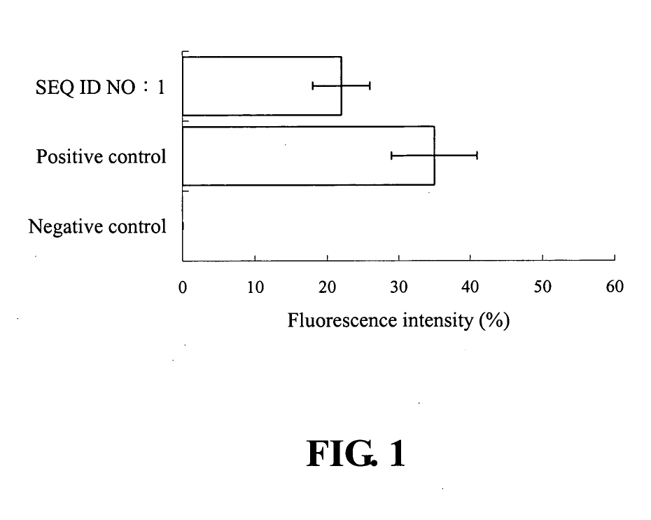

FIG. 1 revealed the results of the T2 cell binding assay of synthetic peptides and HLA-A2.1 antigens. The fluorescence intensity calculated is around 35% in positive control group which showed completely binding ability of peptides and HLA-A2.1 antigens. The fluorescence intensity is around 22% in experimental group of SEQ ID NO: 1 which showed relatively binding ability of SEQ ID NO: 1 peptides and HLA-A2.1 antigens.

### EXAMPLE 2

### In Vivo Induction of CD8 T Cells Proliferation

4-6 week-old HLA-A2.1 transgenic C57BL/6 mice were immunized with 100 μl containing 100 μg of SEQ ID NO: 1 peptide in adjuvant at 0.2 μM of CpG phosphorethioate oligodeoxynucleotide 1826 (CpG ODN 1826) via intramuscular injection three times at one week intervals. Five days after the third immunization, the splenocytes of mice were recovered and the numbers of CD8⁺IFN-γ⁺ double-positive cells were measured using flow cytometry analyses.

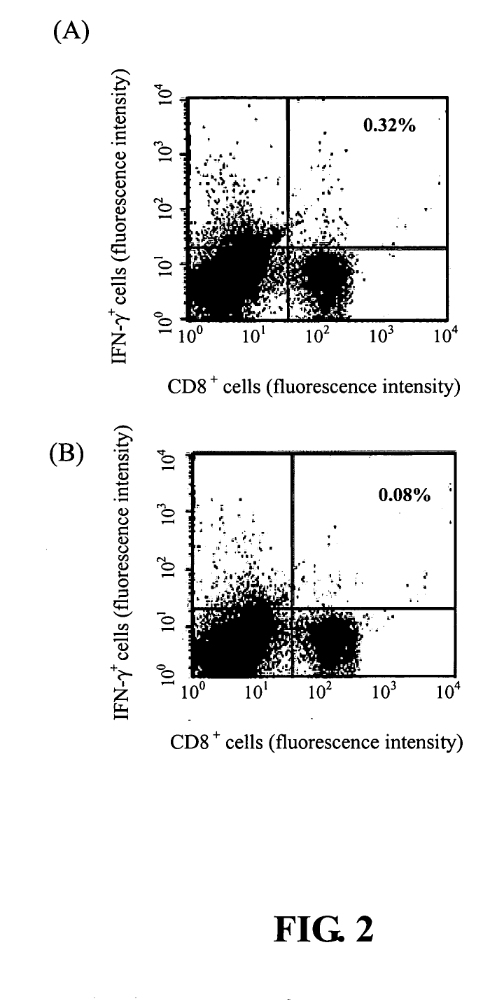

As shown in FIG. 2, the number of CD8⁺IFN-γ⁺ double-positive cells in SEQ ID NO:1 peptide injected mice was 0.32%, while the number was 0.08% in the control group. It is about 4 fold increases with SEQ ID NO: 1 peptide immunized mice in comparison to the irrelevant stimulator. Therefore, SEQ ID NO: 1 peptide is a viral antigen inducer for proliferation of CD8 lymphocytes.

### EXAMPLE 3

### Cytotoxicity Assay

The β-lymphocyte (HMy2.CIR, ATCC No.: CRL-1993) target cells were labeled with PKH-26 and carboxyfluorescein diacetate succinimidyl ester (CFSE). 4-6 week-old HLA-A2.1 transgenic C57BL/6 mice were immunized as described in Example 2 with 100 μl of the same dosage of SEQ ID NO: 1 peptide in adjuvant of CpG oligodeoxynucleotide via intramuscular injection three times at one week intervals. Five days after the third immunization, the splenocytes of mice were recovered as effector cells and incubated with the target cells without labeling.

Target cells labeled with two dyes were cultivated in the absence of effector cells as the control group. When membrane damage occurs, the dye is almost instantaneously lost and the cells are no longer able to take up the charged dye. Hence after cytolysis, the total number of double stained target cells subtracted from the remaining is counted for specific T cells cytolysis.

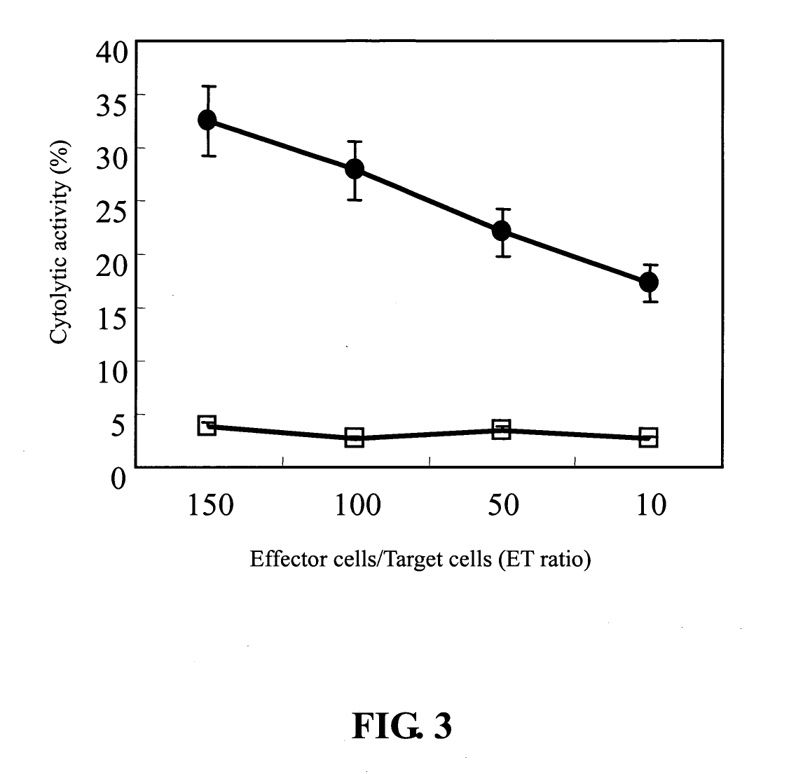

FIG. 3 revealed the CTL activities of SEQ ID NO: 1 peptide at various effector-to-target cell ratio (termed ET ratio thereafter). It was found that the cytolytic activity reached 33% when ET ratio was 150, and the cytolytic activity reached 18% when ratio was 10. Therefore CD8 lymphocyte proliferation induced by SEQ ID NO: 1 peptide can kill the infected cells effectively.

In summary, the peptide antigen of HPV-16 comprising SEQ ID NO: 1 peptide in the invention indeed bound to HLA-A2 class I MHC molecule, transported to the cell surface of antigen presenting cells or infected cells after binding, was further recognized by CTLs and stimulated their proliferation, and then killed the infected, E5-comprising cells to prevent cancer to occur. Therefore, the SEQ ID NO: 1 peptide of HPV-16, which was used as a vaccine, not only increases the numbers of CTLs specific to viral antigens, but also generates peptide-specific CTLs that kill HPV-16 E5 protein-containing cells in an antigen-specific manner in the early-stage of cervical cancer. This will greatly help the prevention and control of early-stage cervical cancers.

What is claimed is:

1. A HPV-16 peptide antigen comprising a peptide sequence of SEQ ID NO: 1, which is able to bind with MHC class I antigen and then is presented on the surface of cell infected by the HPV-16.

2. The antigen as claimed in claim 1, wherein MHC class I antigen is Human Leukocyte Antigen-A2.1 (HLA-A2.1).

3. The antigen as claimed in claim 1, wherein said peptide is able to induce cytotoxic T lymphocytes (CTLs) proliferation.

4. The antigen as claimed in claim 1, wherein said peptide is able to stimulate the CTL responses in cytolysis of cells.

5. The antigen as claimed in claim 1, wherein said antigen is a peptide antigen of HPV-16 E5 protein.

6. A HPV-16 vaccine for preventing and controlling infection of HPV-16, comprising the HPV-16 peptide antigen as claimed in claim 1.
